(19) United States
(12) Patent Application Publication
(10) Pub. No.: US 2014/0271727 A1
(43) Pub. Date: Sep. 18, 2014

(54) METHOD OF USING AN ANTIDEPRESSANT FOR INCREASING IMMUNITY OF A SUBJECT AND TREATING CANCER

(71) Applicant: NATIONAL YANG-MING UNIVERSITY, Taipei City (TW)

(72) Inventors: Jeng-Jong Hwang, Taipei (TW); Chun-Kai Fang, Taipei (TW)

(21) Appl. No.: 13/846,753

(22) Filed: Mar. 18, 2013

(51) Int. Cl. A61K 31/55

(52) U.S. Cl. CPC A61K 31/55
USPC 424/278.1

(57) ABSTRACT

A method of increasing immunity of a subject by administering an antidepressant to the subject is disclosed in the present invention. Preferably, the antidepressant is mirtazapine. And further, another object of the present invention is related to a method of administering the abovementioned antidepressant to the subject for treating cancer. The present invention tries to find the mechanism of the tumor growth inhibition by mirtazapine, and it may be due to the alteration of the tumor microenvironment, which involves the activation of the immune response and the recovery of serotonin level.

### FIELD OF THE INVENTION

This invention relates to a method of using an antidepressant for increasing immunity of a subject, especially relates to a method of using mirtazapine to increasing immunity of the subject with at least a cancer for treating the cancer.

### BACKGROUND OF THE INVENTION

Cancer, known medically as a malignant neoplasm, is a broad group of various diseases, all involving unregulated cell growth. In cancer, cells divide and grow uncontrollably, forming malignant tumors, and invade nearby parts of the body. The cancer may also spread to more distant parts of the body through the lymphatic system or bloodstream. Not all tumors are cancerous. Benign tumors do not grow uncontrollably, do not invade neighboring tissues, and do not spread throughout the body. There are over 200 different known cancers that afflict humans.

Due to the uncertainty of cancer, up to 1 in 4 people with cancer have clinical depression. Clinical depression causes great distress, impairs functioning, and might even make the person with cancer less able to follow their cancer treatment plan. However, the clinical depression can be treated by the antidepressant, and that treatments can reduce suffering and improve their quality of life.

Some serotonin reuptake inhibitors (hereafter called “SSRIs”) and tricyclic antidepressants contribute to the successful antidepressant therapy mainly through decreasing the production of pro-inflammatory cytokines, such as IFN-γ, and increasing the anti-inflammatory cytokines. Nevertheless, it remains unclear whether immune response plays a causative role in the pathophysiology of depressive disorders. The increased sIL-12 levels in patients with major depressive disorders have been reported to be decreased after the treatment with antidepressants, including nefazodone, paroxetine, fluoxetine, sertraline, and venlafaxine. sIL-12, a multifunctional cytokine, is recognized as a key regulator for the cell-mediated immune response. Preclinical trials show that the immunomodulatory and anti-angiogenic functions of sIL-12 are through the activation of innate cells (NK and NK-T cells) and adaptive immune response (CD4+ and CD8+ T cells), priming the secretion of IFN-γ. The antitumor effect of sIL-12 in patients treated with continuous administration of antidepressants, however, is gradually reduced and limits its clinical application. On the other hand, the IFN-γ levels in the whole bloods obtained from healthy volunteers were inhibited when treated with antidepressants.

### SUMMARY OF THE INVENTION

In addition to the use of the antidepressant as mentioned above, the present invention discloses a method of increasing immunity of a subject by using the antidepressant. The method comprises a step of administering an antidepressant to the subject. Preferably, the antidepressant is mirtazapine.

Preferably, the subject is a patient with at least a cancer. And then, the method disclosed in the present invention can further inhibit the tumor growth.

Preferably, the antidepressant increases the concentration of cytokine within the subject. Preferably, the cytokine at least comprises IL-12.

Preferably, the antidepressant increases the concentration of serotonin within the subject.

Another object of the present invention is to provide a method of using an antidepressant for treating cancer, and the method comprises a step of administering the antidepressant to a subject. Preferably, the antidepressant is mirtazapine.

Preferably, the method disclosed in the present invention for treating cancers is achieved by increasing immunity of the subject.

Preferably, the subject is a patient with at least a cancer, and the cancer can be choose from a group consisting of squamous cell carcinoma, lobular carcinoma in situ, liver cancer, nasopharyngeal carcinoma, lung cancer, bone cancer, pancreatic cancer, skin cancer, head and neck cancers, malignant melanom, cervical cancer, ovarian cancer, colon cancer, anal cancer, stomach cancer, breast cancer, testicular cancer, fallopian tube cancer, endometrial cancer, cervical cancer, vaginal cancer, vulvar cancer, non-Hodgkin lymphoma, Hodgkin lymphoma, esophageal cancer, thyroid cancer, adrenal cancer, cancers of mesothelial and soft tissue, urethra cancer, cancer of penis, prostate cancer, acute leukaemia, chronic leukaemia, lymphomas, bladder cancer, ureteral cancer, renal cell carcinoma, urothelial carcinoma, cancer of central nervous system, primary central nervous system lymphoma, glioma, pituitary tumor, Kaposi's sarcoma, squameous cell cancer and their metastasis.

Preferably, the antidepressant increases the concentration of cytokine within the subject, and the cytokine preferably comprises IL-12. And then, the concentration of IFN-γ within the subject will be increased by the induction of IL-12.

Preferably, the antidepressant increases the amount of CD4+ and CD8+ T cells.

Preferably, the antidepressant inhibits the production of TNF-α within the tumor.

Preferably, the frequency of administering the antidepressant to the subject is once a day prior to night sleep, and a dose of the antidepressant is 7.5-60 mg/day. Preferably, 30 mg/day before the night sleep.

The features and advantages of the present invention will be understood and illustrated in the following specification and FIGS. 1A˜7.

### BRIEF DESCRIPTION OF THE DRAWINGS

FIG. 1A to FIG. 1B are diagrams showing the experimental designs according to an embodiment of the present invention;

FIG. 2A to FIG. 2B are diagrams showing effects of mirtazapine (10 mg/kg/d) on behavior changes of normal and CT26/luc tumor-bearing mice;

FIG. 3A to FIG. 3G are diagrams showing that mirtazapine inhibits tumor growth and prolongs the survival rate and interval in CT-26/luc tumor-bearing model;

FIG. 4A to FIG. 4D are diagrams showing immunocompetence analysis in CT26/luc-bearing mice;

FIG. 5 is diagrams showing immunohistostaining of infiltrating CD4+ and CD8+ T cells in tumor tissues of mirtazapine-treated;

FIG. 6A to FIG. 6B are diagrams showing effects of mirtazapine on TNF-α expressions in the blood circulation and tumors; and

FIG. 7 is a diagram showing serotonin transporter determined with [¹²³I]ADAM/ex vivo autoradiography in the brain of CT26/luc tumor-bearing mice.

### DETAILED DESCRIPTION OF THE INVENTION

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

As used herein, the symbol “+” means that the cell surface marker expresses on the surface of the cells and has a larger expressed amount measured by flow cytometer than that of the negative control.

Preferably, all abovementioned expressed amount of the cell surface markers are measured by flow cytometer, however, the present invention is not limited thereto.

As used herein, the term “Interleukin” or “IL” means a group of cytokines that were first seen to be expressed by white blood cells (leukocytes). It has since been found that interleukins are produced by a wide variety of body cells. The function of the immune system depends in a large part on interleukins.

As used herein, the term “BALB/c” is an albino, laboratory-bred strain of the House Mouse from which a number of common substrains are derived. Now over 200 generations from New York in 1920, therefore, BALB/c mice are distributed globally, and are among the most widely used inbred strains used in animal experimentation.

As used herein, the term “SCID” is a genetic disorder in which both B cells and T cells of the adaptive immune system are impaired due to a defect in one of several possible genes.

As used herein, the term “CD4” is a glycoprotein found on the surface of immune cells such as T helper cells.

As used herein, the term “CD8” is a transmembrane glycoprotein that serves as a co-receptor for the T cell receptor (TCR). Like the TCR, CD8 binds to a major histocompatibility complex (MHC) molecule, but is specific for the class I MHC protein.

As used herein, the term “IFN-γ” is a dimerized soluble cytokine that is the only member of the type II class of interferons. And further, it is critical for innate and adaptive immunity against viral and intracellular bacterial infections and for tumor control.

As used herein, the term “TNF-α” means tumor necrosis factor, and it is a cytokine involved in systemic inflammation and is a member of a group of cytokines that stimulate the acute phase reaction. It is produced chiefly by activated macrophages (M1), although it can be produced by many other cell types as CD4+ lymphocytes, NK cells and neurons.

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention. To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Preferably, the results as shown in FIG. 4C and FIG. 4D are performed by a flow cytometer, and a target cell population will be screened out by utilizing at least one flow cytometer to identify different surface markers of different cells. Flow cytometry allows for single cell analysis at speeds far surpassing any other single cell analysis technology in the art. This enables a statistically significant number of cells to be analyzed faster than using other alternative techniques. In a preferred embodiment, a flow cytometer is used with any suitable sample preparation robot or liquid handler that is known in the art. Furthermore, a single laser flow cytometer is used in an embodiment for the analyzing step. In another embodiment, a multi-laser flow cytometer is used for the analyzing step and the present invention is not limited thereto.

As mentioned above, the antidepressants are prescribed for the treatment of patients with depression. Mirtazapine, one of the antidepressant, is a noradrenergic and specific serotonergic antidepressant (NaSSA) which was introduced by Organon International in the United States in 1990 and is used primarily in the treatment of depression. It is also commonly used as an anxiolytic, hypnotic, antiemetic, and appetite stimulant. Structurally, mirtazapine can also be classified as a tetracyclic antidepressant (TeCA). Mirtazapine is also an antagonist for the adrenergic alpha2-autoreceptors and alpha2-heteroreceptors with its high affinity for both 5-HT3 and 5-HT2A receptors.

In clinical treating, Applicant found that mirtazapine may be effective for improving multiple symptoms, including cachexia, anorexia, and quality of life in patients with advanced cancer. However, other kinds of the antidepressant have no such effect. Therefore, Applicant tries to establish a CT26/luc colorectal carcinoma-bearing animal model combined with molecular imaging in the present invention to proof the effect of mirtazapine on tumor growth inhibition and its correlation with tumor microenvironment.

The details in preparing the materials and processing relative analyses will be illustrated as follows. First, CT-26 murine colon carcinoma cells (obtained from Taiwan Liposome Company, Taipei, Taiwan) were transfected with the luciferase gene (hereafter called “CT26/luc”). The CT26/luc tumor cells were cultured in RPMI 1640 medium (Invitrogen) supplemented with 10% fetal bovine serum (Hyclone), 100 units/ml of penicillin, and 100 mg/ml streptomycin (Gibco-BRL) at 37° C. in a 5% CO₂ atmosphere.

Before starting the following investigation. The cell viability is analyzed. 3-(4,5-Dimethylthiazol-2-yl)-2,5-diphenyltetrazolium bromide (MTT, Sigma, USA) was dissolved in phosphate-buffered saline (145 mM NaCl, 1.4 mM KH₂PO₄, 4.3 mM Na₂HPO₄, and 2.7 mM KCl, pH 7.2). The CT26/luc cells were seeded in 96-well plates overnight, and then treated with various concentrations, such as 0, 5, 10, 20, 40, and 80 μM, of mirtazapine for 24, 48, and 72 hrs. And then, cell viability was determined with MTT assay. After washing with fresh medium, 100 μl of 1 mg/ml MTT solution was added to each well. After 4 hours incubation at 37° C., 100 μl DMSO was added to dissolve the MTT formazan, and the absorbance was determined with an ELISA reader (Power Wave X340, Bio-Tek Instrument Inc., USA) using a wavelength of 570 nm for the excitation. The CT26/luc cells were cultured in 10 cm-diameter dish (1×10⁶/dish) for 24 hrs, followed by the treatments with 0, 5, 10, 20, 40, and 80 μM mirtazapine (Megafine Pharma (P) Ltd., India). The CT26/luc cells were harvested in 15 ml centrifuge tubes 24 hrs later, fixed with cold 75% alcohol overnight. The CT26/luc Cells were then centrifuged at 5000 rpm for 15 min at 4° C. After removal of the supernatant, the CT26/luc cells were resuspended in 0.8 ml cold phosphate-buffered saline (PBS), 0.1 ml RNase A (1 mg/ml, QIAGEN), and 0.1 ml propidium iodide (400 μg/ml) for 30 min at 37° C. and kept in the dark to avoid quenching. The cell cycle analysis was assayed using a FACScan (BD Sciences) and analyzed by CellQuest software (BD Sciences).

Second, all animal study protocols were approved by the Institutional Animal Care and Use Committee (IACAU) of National Yang Ming University. Mirtazapine (0.25 mg) was dissolved in 0.05 ml of 0.9% NaCl plus 0.5% absolute ethanol for each mouse (i.e. 10 mg/kg.) Male BALB/c mice (initial weights 25±2 g, and purchased from the National Laboratory Animal Center, Taipei, Taiwan) were housed in the cages, five mice per cage, under a 12:12 h reverse light/dark cycle with lights off at 6 pm Animals were handled and weighed daily for a week to reduce any non-specific stress responses. And then, please refer to FIG. 1A and FIG. 1B, FIG. 1A and FIG. 1B are diagrams showing the experimental designs according to an embodiment of the present invention. As shown in FIG. 1A, 6-weeks-old male BALB/c mice were randomly divided into 6 groups, such as the group named “wild-type” (hereafter called “wide-type”), the group named “drug” (hereafter called “drug”), the group named “never” (hereafter called “never”), the group named “always” (hereafter called “always”) and the group named “after” (hereafter called “after”). “Wild-type” means the mice are monitored without tumor inoculation and mirtazapine treatment, drug means the mice are monitored with continuous mirtazapine treatment but without tumor inoculation, never means the mice are monitored with tumor inoculation and daily 0.05 ml of 0.9% NaCl plus 0.5% absolute ethanol treatment but without mirtazapine treatment, always means the mice are monitored with mirtazapine treatment initiated 2 weeks before the tumor inoculation, and concurrent means the mice are monitored with tumor inoculation and mirtazapine treatment on the same day, and after means the mice are monitored with mirtazapine treatment initiated 2 weeks post the tumor inoculation. Furthermore, the experimental design and the time for the biological end points were shown in FIG. 1B. That is, the mice were assayed on day 22 for behaviors, and then sacrificed for the measurement of lymphocyte subsets and performed with ex vivo autoradiography.

Accordingly, the CT26/luc cells (2×10⁶ cells/200 μL) suspended in the serum-free RPMI medium were transplanted subcutaneously into the dorsal region of the right thighs of the BALB/c mice. 10 mg/kg/d mirtazapine dissolved in 0.9% sodium chloride and 0.5% ethanol was administered to mice by gavage daily till mice expired or terminated on day 67 post tumor inoculation. Survival rate and interval were assayed for never, always, concurrent, and after (n=10 per group).

Also, Applicant divides other six-weeks-old immunodeficient male SCID mice (purchased from the National Laboratory Animal Center, Taiwan) into two groups, such as the group named “never-SCID” (hereafter called “never-SCID”) and the group named “always-SCID” (hereafter called “always-SCID”), as control groups for verifying the involvement of the immune system in the inhibition of the tumor growth by mirtazapine. Never-SCID means that the mice are monitored with tumor inoculation but without mirtazapine treatment, and always-SCID means the mice are monitored with mirtazapine treatment initiated 2 weeks before tumor inoculation.

As to the tumor growth, it was monitored using a digital caliper twice a week. The tumor volume was calculated according the following formula:

0.5236×length×width×thickness

Bioluminescence imaging (hereafter called “BLI”) used for tumor size tracking was performed with an IVIS50 animal imaging system (Xenogen Corp., USA).

The behavioral change in the animal depression model was evaluated and the mouse was placed for the spontaneous motor activity assay in a separate chamber and allowed to rest for 3 min. The number of movements was automatically counted during a 5-min period (Process Control, ActiMot 302020, TSE Systems). On the other hand, the duration of immobility was assayed with the tail suspension test. Acoustically and visually isolated mouse was suspended at the tip of the tail with 50 cm high above the floor. Immobility time was recorded for 6 min.

As to the quantification of interleukin-12 (hereafter called “IL-12”), the whole blood withdrawn from the pouch of each mouse was centrifuged at 600×g for 20 min, and serum was collected. The serum IL-12p70 (sIL-12) level was determined using an ELISA kit (R&D Systems, Taiwan). Identification for the lymph node cluster of differentiated CD4+ T helper and CD8+T-cytotoxic lymphocyte subsets was assayed. Briefly, the lymphocytes isolated from the lymph nodes of groins of mice were stained with phycoerythrin-conjugated antimouse CD4 (CD4-PE) monoclonal antibody and peridininchlorophyll-protein-complex-conjugated anti-mouse CD8 (CD8-PerCP) monoclonal antibody (BioLegend, USA). Lymphocyte subsets were identified by FACS analysis using a FACS Calibur flow cytometer. Immunohistochemistry (IHC) of CD4 and CD8 was also performed on day 42 post the tumor inoculation. Tumors were then removed, paraffin embedded, and 5-mm sectioning was performed. The sections were immunohistostained with antibodies against CD4 (BioLegend, USA) and CD8 (BioLegend, USA), respectively. The procedures of immunohistostaining were followed the protocols provided with the IHC kit (Millipore, USA). All images were digitally captured on a Scanscope CS system (Aperio, USA).

And then, the level of IFN-γ in the tumor was determined using an ELISA kit (R&D Systems, Taiwan). Briefly, 6 weeks after tumor inoculation, the mice were sacrificed and the tumors were quickly removed and minced, then added with lysis buffer containing 1% protease inhibitor cocktail (T-PER tissue protein extraction reagent, Thermo Scientific, USA). After sonication, the cell mixture was centrifuged with 15000 rpm (Kubota centrifuge 1700, Japan) at 4° C. for 10 min The supernatant was collected for the protein quantification using bovine serum albumin as the standard. Two mg of the tumor proteins was used for the quantification of IFN-γ.

As to the quantification of TNF-α, the whole blood withdrawn from the pouch of each mouse once a week for up to 6 weeks was centrifuged at 600×g for 20 min, and serum was collected. The serum TNF-α level was evaluated with an ELISA kit (eBioscience, USA). The level of TNF-α in the tumor of mice on day 42 post tumor inoculation was determined using ex vivo Western Blotting assay. Briefly, 6 weeks after tumor inoculation, the mice were sacrificed and the tumors were quickly removed and minced, then added with lysis buffer containing 1% protease inhibitor cocktail (T-PER tissue protein extraction reagent, Thermo Scientific, USA). Equal amounts of proteins (40 μg) were subjected to SDS-PAGE and transferred to PVDF membranes (Millipore, Bedford, Mass.). Non-specific binding was blocked by incubation with 5% non-fat milk. Membrane was incubated with antibodies against TNF-α (Abbiotec, USA) and β-actin (Millipore, USA) overnight at 4° C. The goat-anti rabbit IgG (Millipore) and goat-anti mouse IgG conjugated with horseradish peroxidase (Millipore) were used as the secondary antibodies. The band signal from the antigen-antibody binding was illustrated with enhanced chemoluminescence system (ECL, Millipore). Image J software (National Institutes of Health, USA) was used for the quantitative analysis.

As to the uptake of 2-((2-((dimethylamino)methyl)phenyl)thio)-5-iodophenylamine (it is purchased from the Institute of Nuclear Energy Research, Taiwan, and hereafter called [¹²³I]ADAM) in the moue brain was. The CT26/luc tumor-bearing mice were injected with 1 mCi/0.1 ml of [¹²³] ADAM via the caudal vein, and sacrificed at 90 min post injection, and assayed with ex vivo autoradiography. Briefly, the brain slices (5 mm thickness) were put onto an imaging plate (BAS cassette 2340, FujiFilm, Japan), and exposed for 24 hours. The imaging plates were then scanned with a high-resolution imaging plate reader (FLA5000, FujiFilm, Japan) at the following settings: resolution 25, gradation 16 bits, and dynamic range L5. The specific binding ratio (SBR) was calculated as the following: SBR=(target−cortex)/cortex.

The final analysis is a statistical analysis. That is, all data were shown as the mean±standard error. Student's test was used for the comparison between two groups. Kaplan-Meier plotting was used for the survival analysis, and was compared using the log-rank test. Differences between the means were considered significant if p<0.05 or less.

According to all abovementioned steps, assays and analyses, it is clearly that mirtazapine can increase the immunity of the mice for further treating cancer. First, the spontaneous motor activity and immobility time of mice were evaluated on day 22 after tumor inoculation and with or without mirtazapine intervention, and the effects of mirtazapine (10 mg/kg/d) on behavior changes of normal and CT26/luc tumor-bearing mice are shown in FIG. 2A and FIG. 2B. As shown in the figures, the increase in the immobility time and the decrease in the number of spontaneous motor activity were observed after the implantation of the CT26/luc tumors as shown with never. Continuous administration of mirtazapine significantly decreased the immobility time, but had no effect on the spontaneous motor activity as shown with drug and always.

Please refer to FIGS. 3A-3B, FIG. 3A to FIG. 3G are diagrams showing that mirtazapine inhibits tumor growth and prolongs the survival rate and interval in CT-26/luc tumor-bearing model. As shown in FIG. 3A, significant tumor growth inhibition (p<0.01) was found in all mirtazapine-treated groups, such as always, concurrent and after, as compared to that of never from day 22-47 after the tumor inoculation. And further, tumor growth delay of the always was significant higher than those of the concurrent and after groups (p<0.01). BLI also confirmed the similar results as shown in FIG. 3B and FIG. 3C. The tumor inhibition effect of mirtazapine, however, was not found in the SCID mice as shown in FIG. 3D and FIG. 3E. In addition, no significant body weight change throughout the experiment was found among all groups indicated no general toxicity with mitazapine treatment as shown in FIG. 3F. The overall survival times for mirtazapine-treated, tumor-bearing mice (that is, always, concurrent and after) were then all significantly longer than that of never (43.1±2.6 days) as shown in FIG. 3G The survival times for always, concurrent, and after were 66.91±0.1, 63.61±1.5, and 57.01±3.2 days, respectively. The survival time of always was significantly longer than that of the concurrent (p<0.01) as shown in FIG. 3E. Please refer to Table 1 as shown in the following wherein the mean tumor growth time means the time at which the tumor volume reaches to 400 mm, the mean tumor growth delay time means the tumor growth time of the treated group minus that of never, and the mean growth inhibition rate means the growth rate of the treated group/the growth rate of never:

TABLE 1

| | | | mean tumor | mean tumor |
|---|---|---|---|---|
| | | mean tumor | growth delay | growth inhibition |
| Group | n | growth time | time | rate |
| Never | 12 | 22.5 | N/A | N/A |
| Always | 12 | 41.3 | 18.8 | 1.8 |
| Concurrent | 12 | 30.9 | 8.2 | 1.4 |
| after | 12 | 25.4 | 2.8 | 1.1 |

In Table 1, mice treated with mirtazapine two weeks prior to the tumor inoculation, that is always, showed the highest inhibition of tumor growth.

Please further refer to FIGS. 4A˜4D, FIG. 4A shows that sIL-12 concentrations are increased to the peak levels with 13 and 18 folds at 0 and 1 wk post tumor cell inoculations for drug and always, respectively. On the other hand, sIL-12 concentrations were increased 17, 16 and 13 folds for concurrent, after and never, respectively. Notably, the sIL-12 concentration of never returns to the normal level, but drug still remains high (42 vs. 7 pg/ml) at 4 wks post tumor cell inoculation. The results suggest that the effect of tumor growth on sIL-12 level is less than that of continuous mirtazapine treatment, especially when drug administration is prior to tumor inoculation. The sIL-12 concentrations of always and concurrent were still significantly higher than that of after, the latter dropped to the control level at 6 weeks post tumor inoculation (p<0.01 and p<0.05, respectively). The increase of sIL-12 level after mirtazapine treatment, however, was not found in the SCID mice as shown in FIG. 4B.

In addition, both CD4+ and CD8+ T cell counts were lower in CT26/luc tumor-bearing mice (never), but not in the mirtazapine-treated, tumor-bearing mice (always, concurrent, and after) as compared with those of wild type and drug in Table 2.

TABLE 2

| | CD4+ T cells | CD8+ T cells |
|---|---|---|
| Group | (10⁴ events) | (10⁴ events) |
| Wild-type | 32.63 ± 1.36% | 28.80 ± 7.00% |
| Drug | 30.97 ± 1.40% | 30.95 ± 6.57% |
| Never | 17.49 ± 1.07% | 12.76 ± 3.10% |
| Always | 29.75 ± 1.96% | 32.77 ± 7.43% |
| Concurrent | 25.77 ± 0.73% | 22.41 ± 5.03% |
| after | 22.58 ± 1.15% | 15.86 ± 4.78% |

As shown in FIG. 4C and FIG. 4D, FIG. 4C shows that CD4 PE vs. CD3 FITC T lymphocytes and FIG. 4D shows that CD8-PerCP vs. CD3 FITC T lymphocytes. Both FIG. 4C and FIG. 4D are determined with the flow cytometer and also tabulated in Table 2 as above. It is clearly that both CD4+ and CD8+ T cell counts of always were the highest among the three mirtazapinetreated, tumor-bearing animal. As to the expression of IFN-γ in tumors is listed in Table 3, and it was significantly higher in always, concurrent, and after as compared with that of never, with the highest expression in always. In addition, earlier mirtazapine intervention, such as always and concurrent, resulted in significantly higher IFN-γ expression as compared with that of after.

TABLE 3

| Group | IFN-γ (pg/ml) |
|---|---|
| Never | 4.15 ± 0.25 |
| Always | 85.35 ± 4.5 |
| Concurrent | 39.42 ± 7.42 |
| after | 19.60 ± 1.13 |

Please refer to FIG. 5A and FIG. 5B, it notably shows that significantly increased numbers of infiltrating CD4+ and CD8+ cells/0.1 mm² tumor tissues of “concurrent” and “always” as compared with those of “never”, and were quantified in FIG. 5C, p<0.01 and p<0.001, respectively.

Please refer to FIG. 6A and FIG. 6B, the serum TNF-α level was evaluated with enzyme-linked immunosorbent assay (ELISA) once a week for up to 6 weeks post tumor inoculation. The serum TNF-α levels are gradually increased from the third weeks up to six weeks post tumor inoculation as shown in FIG. 6A, however, no significant difference is found among tumor-bearing mice treated with and without mirtazapine, respectively. On the other hand, the TNF-α levels in tumors of mice (Always, Concurrent, and After) assayed with ex vivo Western blotting on day 42 post tumor inoculation were decreased to 40% of that of “Never” as shown in FIG. 6B.

The higher uptake of [¹²³I] ADAM by serotonin transporter (SERT)-rich areas, such as olfactory tubercle, lateral septal nucleus, thalamic nuclei, substantia nigra, and hypothalamic nuclei, in the brain is shown in FIG. 7 as determined with ex vivo autoradiography. The specific binding ratios (SBRs) of [¹²³I] ADAM in SERT-rich areas of mice are listed in Table 4, in which specific binding ratio=(target−cortex)/cortex. SBRs were significantly higher in drug as compared with those of wild type (p<0.05). SBRs in always, concurrent, and after were also significantly higher than those of never (p<0.05). The results are in accordance with that SERT-rich areas are more susceptible to mirtazapine treatment. In addition, earlier mirtazapine intervention, as always and concurrent, contributes to a more significant increase of SBRs as compared with that of after (p<0.01).

TABLE 4

| | Specific binding ratio | | | | |
|---|---|---|---|---|---|
| Group | LS | OT | ThN | SN | HN |
| Wild-type | 1.45 ± 0.05 | 1.36 ± 0.10 | 1.23 ± 0.14 | 2.58 ± 0.10 | 1.55 ± 0.12 |
| Drug | 1.77 ± 0.10 | 1.95 ± 0.16 | 1.72 ± 0.09 | 2.81 ± 0.08 | 2.14 ± 0.12 |
| Never | 1.13 ± 0.07 | 1.12 ± 0.06 | 0.93 ± 0.15 | 1.47 ± 0.07 | 1.28 ± 0.12 |
| Always | 2.00 ± 0.04 | 2.01 ± 0.08 | 1.99 ± 0.07 | 2.46 ± 0.07 | 2.29 ± 0.10 |
| Concurrent | 1.78 ± 0.05 | 1.91 ± 0.09 | 1.76 ± 0.07 | 2.44 ± 0.06 | 2.227 ± 0.132 |
| after | 1.36 ± 0.03 | 1.63 ± 0.12 | 1.47 ± 0.06 | 2.17 ± 0.16 | 2.11 ± 0.12 |

According to the abovementioned results, Applicant found that in vivo chronic mirtazapine treatment could inhibit the tumor growth and prolong the survival of tumor-bearing mice, which showed increased serum IL-12 level, CD4+, CD8+ in the lymph nodes, as well as serotonin transporters in the brain, and decreased TNF-α and IFN-γ in the tumors. The increased sIL-12 levels in mirtazapine-treated mice are maintained above the pre-therapy levels for more than four weeks, especially those with early mirtazapine intervention, such as always which show the highest survival rate and time with the highest increase of sIL-12 levels and the uptake of [¹²³I]ADAM, a radiophamaceutical for serotonin transporter. Immunodeficient mice, on the other hand, do not show the similar effects when treated with mirtazapine. Both CD4+ and CD8+ T cells, may also contribute to the anticancer effect since their counts are recovered in those tumor-bearing mice treated with mirtazapine as shown in Table 2. The IFN-γ levels in tumors of mice treated with mirtazapine are significantly higher than those untreated, suggest that the immune response may be also involved in the antitumor effect of mirtazapine.

The present invention further shows that mirtazapine is nontoxic to CT26 colon and the plasma levels of TNF-α and soluble TNF receptors are increased in patients with major depressive disorders treated with mirtazapine. With norepinephrine transporter knockout mice found that the decrease of IL-6 and IFN-γ, and the increase of IL-4 production may be due to the increase of norepinephrine level in the spleen after mirtazapine treatment. On the other hand, IFN-γ-indoleamine 2,3-dioxygenase (IDO) axis also has been reported to regulate the sIL-12-mediated antitumor immunity, in which IFN-γ is the main cytokine induced by sIL-12 and plays a critical role to its antitumor effects. IDO is highly inducible by pro-inflammatory cytokines, including IFN-γ and tumor necrosis factor-α (TNF-α). IDO is the first and rate-limiting enzyme involved in the tryptophan-kynurenine pathway. Degradation of tryptophan through the kynurenine pathway shows important neuropsychiatric implications. In addition, IDO is expressed in the brain so that fluctuations in its enzymatic activity can affect serotonin biosynthesis. Decreased tryptophan concentration affects the serotonergic neurotransmission in the brain. Therefore, adequate physiological serotonin levels are indispensable for cytokine production. Mirtazapine may have a role in restoration of the equilibrium between physiological and pathological levels of cytokines in the brain.

Furthermore, [¹²³I]ADAM is an useful radiophamaceutical for diagnosing serotonin transporter (SERT) location sites in central nervous system (CNS), peripheral nervous system (PNS), and neuroendocrine tissues/organs, such as mucosa of the stomach and medulla of the adrenal glands. Thus, the SERT-rich regions in the mouse brain can also be determined with ex vivo autoradiography using [¹²³]ADAM. Although only the higher specific SERT binding sites in the midbrain for [¹²³]ADAM with ex vivo autoradiography were shown in FIG. 7, the PNS and neuroendocrine tissues/organs should have the higher uptake of [¹²³]ADAM as well. SERT availability in the midbrain of healthy subjects imaged with [¹²³]ADAM/SPECT has been shown to correlate with the overall rating scores and the life quality. Here, Applicant found that the lower uptake of [¹²³]ADAM in the midbrain of tumor-bearing mice could be recovered when treated with mirtazapine. Since the quality of life can be used as a prognostic factor in cancer patients, its improvement by mirtazapine may also contribute to the overall survival via normal serotonergic activity in the brain of subject.

The present also shows that the most therapeutic efficacy for cancer treatment is “Always”, where the mice are pretreated with mirtazapine, a tetracyclic antidepressant, for two weeks before tumor cell injection. This finding implies that mirtazapine may also exert the similar therapeutic effect on tumor prevention as do those selective serotonin reuptake inhibitors (SSRI). This might also be interpreted as an effect on tumor establishment/prevention, or perhaps that the mirtazapine needs several weeks to take effect if it is an indirect effect on the serotonin and then the cytokines.

In conclusion, it can be proved that the better tumor growth inhibition and the longer survival rate and time are found in tumor-bearing mice treated with mirtazapine, especially in those with early intervention. Thus, the present invention suggests that the antitumor effect of mirtazapine in CT26/luc colon carcinoma-bearing mice is via the activation of the immune response and the recovery of serotonin level in serotonergic system.

To sum up, the present invention provides a method of increasing immunity of a subject by using mirtazapine. If the subject is a patient with at least a cancer, the present invention further provides a method of treating cancer using the same. In other words, the immunity and depression of the subject can be both improved by mirtazapine for further inhibiting the tumor growth. That is, the purpose of the present invention is to provide a new use of mirtazapine, and the inhibition method of the present invention is to help the subject increase his or her own immunity by increasing the concentration of cytokine, such as IL-12 and serotonin And then, the concentration of IFN-γ will be induced to rise by IL-12 for further cancer therapy.

It is noted that the cancer therapy will not be limited to any kind of cancers in the present invention. That is, the cancer can be choose from a group consisting of squamous cell carcinoma, lobular carcinoma in situ, liver cancer, nasopharyngeal carcinoma, lung cancer, bone cancer, pancreatic cancer, skin cancer, head and neck cancers, malignant melanom, cervical cancer, ovarian cancer, colon cancer, anal cancer, stomach cancer, breast cancer, testicular cancer, fallopian tube cancer, endometrial cancer, cervical cancer, vaginal cancer, vulvar cancer, non-Hodgkin lymphoma, Hodgkin lymphoma, esophageal cancer, thyroid cancer, adrenal cancer, cancers of mesothelial and soft tissue, urethra cancer, cancer of penis, prostate cancer, acute leukaemia, chronic leukaemia, lymphomas, bladder cancer, ureteral cancer, renal cell carcinoma, urothelial carcinoma, cancer of central nervous system, primary central nervous system lymphoma, glioma, pituitary tumor, Kaposi's sarcoma, squameous cell cancer and their metastasis.

Although the present invention has been described in terms of specific exemplary embodiments and examples, it will be appreciated that the embodiments disclosed herein are for illustrative purposes only and various modifications and alterations might be made by those skilled in the art without departing from the spirit and scope of the invention as set forth in the following claims.

I claim:

1. A method of increasing immunity of a subject comprising:
administering an antidepressant to the subject, wherein the antidepressant is mirtazapine.

2. The method according to claim 1, wherein the subject is a patient with at least a cancer.

3. The method according to claim 2 further inhibiting the tumor growth.

4. The method according to claim 1, wherein the antidepressant increases the concentration of cytokine within the subject.

5. The method according to claim 4, wherein the cytokine at least comprises IL-12.

6. The method according to claim 1, wherein the antidepressant increases the concentration of serotonin within the subject.

7. A method of administering an antidepressant to a subject for treating cancer, wherein the antidepressant is mirtazapine.

8. The method according to claim 7 being achieved by increasing immunity of the subject.

9. The method according to claim 7, wherein the subject is a patient with at least a cancer.

10. The method according to claim 9, wherein the cancer can be choose from a group consisting of squamous cell carcinoma, lobular carcinoma in situ, liver cancer, nasopharyngeal carcinoma, lung cancer, bone cancer, pancreatic cancer, skin cancer, head and neck cancers, malignant melanom, cervical cancer, ovarian cancer, colon cancer, anal cancer, stomach cancer, breast cancer, testicular cancer, fallopian tube cancer, endometrial cancer, cervical cancer, vaginal cancer, vulvar cancer, non-Hodgkin lymphoma, Hodgkin lymphoma, esophageal cancer, thyroid cancer, adrenal cancer, cancers of mesothelial and soft tissue, urethra cancer, cancer of penis, prostate cancer, acute leukaemia, chronic leukaemia, lymphomas, bladder cancer, ureteral cancer, renal cell carcinoma, urothelial carcinoma, cancer of central nervous system, primary central nervous system lymphoma, glioma, pituitary tumor, Kaposi's sarcoma, squameous cell cancer and their metastasis.

11. The method according to claim 7, wherein the antidepressant increases the concentration of cytokine within the subject.

12. The method according to claim 11, wherein the cytokine at least comprises IL-12.

13. The method according to claim 12, wherein the antidepressant increases the concentration of IFN-γ within the subject by the induction of IL-12.

14. The method according to claim 7, wherein the antidepressant increases the amount of CD4+ and CD8+ T cells.

15. The method according to claim 7, wherein the antidepressant inhibits the production of TNF-α within the tumor.

16. The method according to claim 7, wherein a frequency of administering the antidepressant to the subject is once a day, and a dose of the antidepressant is 7.5-60 mg/day.

17. The method according to claim 16, wherein the dose of the antidepressant is 30 mg/day.
